(19) United States
(12) Patent Application Publication
(10) Pub. No.: US 2009/0149774 A1
(43) Pub. Date: Jun. 11, 2009

(54) BONE MARROW ASPIRATION NEEDLE

(75) Inventors: Bruce J. Simon, Mountain Lakes, NJ (US); John Manocchio, Paramus, NJ (US)

Correspondence Address: HARNESS, DICKEY & PIERCE, P.L.C., P.O. BOX 828, BLOOMFIELD HILLS, MI, 48303, US

(73) Assignee: EBI, L.P., Parsippany, NJ (US)

(21) Appl. No.: 11/951,688

(22) Filed: Dec. 6, 2007

(51) Int. Cl. A61B 10/00

(52) U.S. Cl. 600/567

(57) ABSTRACT

A bone aspiration needle is provided. The cannulated body can define at least a first aperture for aspirating bone marrow from a first region of an anatomy. The bone aspiration needle can also include a second aperture for aspirating bone marrow from a second region of the anatomy and a third aperture for aspirating bone marrow from a third region of the anatomy. The first aperture can be a distal most aperture and the third aperture can be a proximal most aperture. The first aperture can have a first area, the second aperture can have a second area, and the third aperture can have a third area. The second area can be greater than the first area and less than the third area so as to enable the aspiration of substantially equal amounts of bone marrow substantially simultaneously from each of the first aperture, second aperture and third aperture.

### FIELD

The present disclosure relates generally to surgical instruments and procedures, and more specifically, to a method and apparatus for a bone aspiration needle.

### BACKGROUND

The statements in this section merely provide background information related to the present disclosure and may not constitute prior art.

In general, the human musculoskeletal system is composed of a variety of tissues including bone, ligaments, cartilage, muscle, and tendons. Tissue damage or deformity stemming from trauma, pathological degeneration, or congenital conditions often necessitates surgical intervention to restore function. During these procedures, surgeons can use a graft to facilitate the natural healing process.

For example, in the case of damaged bone tissue, a bone graft can be surgically inserted around the damaged bone tissue to facilitate the healing and regrowth of the bone tissue. Bone grafts can comprise allografts, xenografts, autografts, composites, etc., and can typically be held in place with mechanical fasteners until the damaged bone tissue has healed. In some instances, it can be desirable to use materials to speed the healing process, such as bone marrow aspirate. Bone marrow aspirate can be harvested from regions in the body with cancellous bone (i.e. the iliac crest, vertebral bodies, calcaneus, etc.) and the medullary canals of long bones (i.e. the femur, tibia, humerus, etc.), and can be used by surgeons to enhance the healing capabilities of the bone graft.

### SUMMARY

In accordance with one aspect of the present teachings, a bone aspiration needle can include a cannulated body defining at least a first aperture for aspirating bone marrow from a first region of an anatomy. The cannulated body of the bone aspiration needle can also include a second aperture for aspirating bone marrow from a second region of the anatomy and a third aperture for aspirating bone marrow from a third region of the anatomy. The first aperture can be a distal most aperture and the third aperture can be a proximal most aperture. The first aperture can have a first area, the second aperture can have a second area, and the third aperture can have a third area. The second area can be greater than the first area and less than the third area.

In accordance with another aspect of the present teachings, a bone aspiration needle can include a cannulated body that defines at least a distal aperture for aspirating bone marrow from a first region of an anatomy and a proximal aperture for aspirating bone marrow from a second region of the anatomy. The bone aspiration needle can also comprise a suction device that can be in communication with the cannulated body. The suction device can be operable to apply a negative pressure to the cannulated body to aspirate bone marrow from the first region and second region of the anatomy. The distal aperture can have a first area and the proximal aperture can have a second area. The second area can be less than the first area.

In accordance with yet another aspect of the present teachings, a bone aspiration needle can include a cannulated body having a proximal end and a distal end with a tip. The cannulated body can define at least a first aperture for aspirating bone marrow from a first region of an anatomy, a second aperture for aspirating bone marrow from a second region of the anatomy and a third aperture for aspirating bone marrow from a third region of the anatomy. The first aperture can be proximal to the tip, the third aperture can be distal from the tip and the second aperture can be disposed between the first aperture and the third aperture. The bone aspiration needle can also include a suction device. The suction device can be in communication with the proximal end of the cannulated body and operable to apply a negative pressure to the cannulated body to aspirate bone marrow from the first region, the second region and the third region of the anatomy. The first aperture can also have a first area, the second aperture can have a second area and the third aperture can have a third area. The second area can be less than the first area and greater than the third area such that the suction device is operable to aspirate substantially equal amounts of bone marrow from the first aperture, the second aperture and the third aperture.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of a bone aspiration needle according to the present disclosure;

FIG. 2 is an environmental schematic view of the bone aspiration needle of FIG. 1 aspirating an anatomy;

FIG. 3 is a side view of the bone aspiration needle of FIG. 1; and

FIG. 4 is a perspective schematic view of a kit including the bone aspiration needle of FIG. 1.

### DESCRIPTION OF VARIOUS ASPECTS

The following description is merely exemplary in nature and is not intended to limit the present disclosure, application, or uses. It should be understood that throughout the drawings, corresponding reference numerals indicate like or corresponding parts and features. Although the following description is related generally to a bone aspiration needle that can be positioned in a prepared portion of the anatomy, such as in an iliac crest, it will be understood that the bone aspiration needle, as described and claimed herein, can be used in any appropriate surgical procedure. Therefore, it will be understood that the following discussions are not intended to limit the scope of the appended claims.

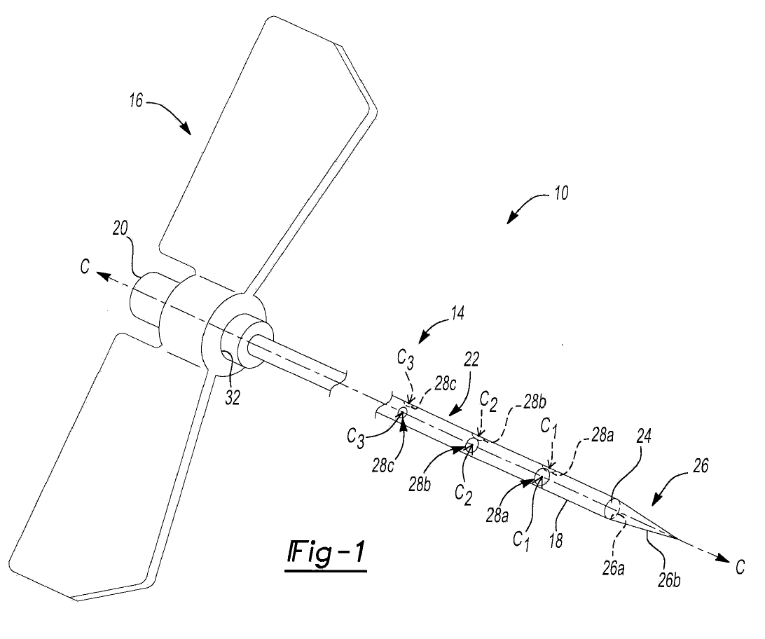

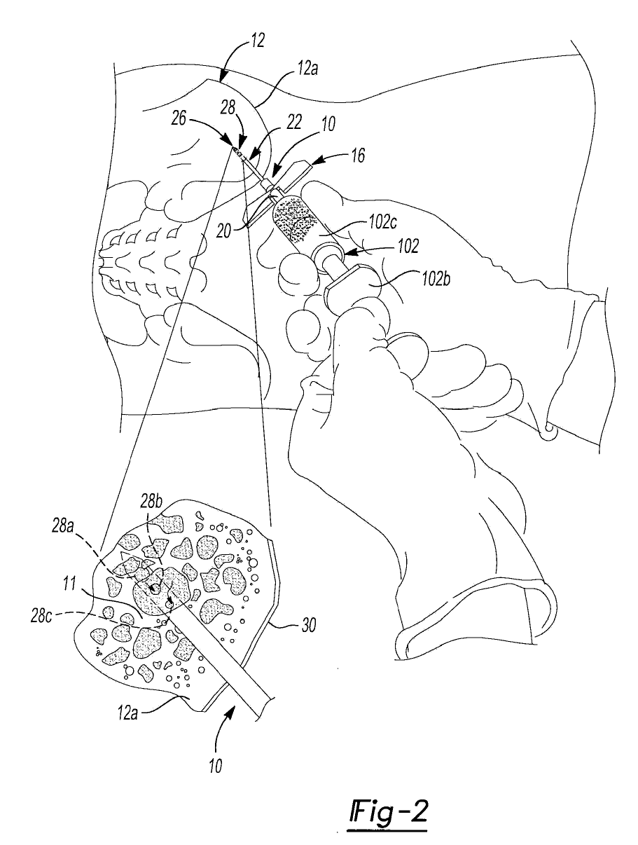

With reference to FIGS. 1 and 2, a bone aspiration needle or bone marrow aspiration needle 10 is shown. With reference to FIG. 2, the bone marrow aspiration needle 10 can improve the extraction of bone marrow aspirate (BMA) 11, generally indicated as 11, from a prepared portion of an anatomy 12, such as an iliac crest 12a, as will be discussed. With reference to FIG. 1, the bone marrow aspiration needle 10 can be formed from a suitable biocompatible material, such as a biocompatible polymer, metal or metal alloy. The bone marrow aspiration needle 10 can include a cannulated body 14 and a graspable portion or handle 16.

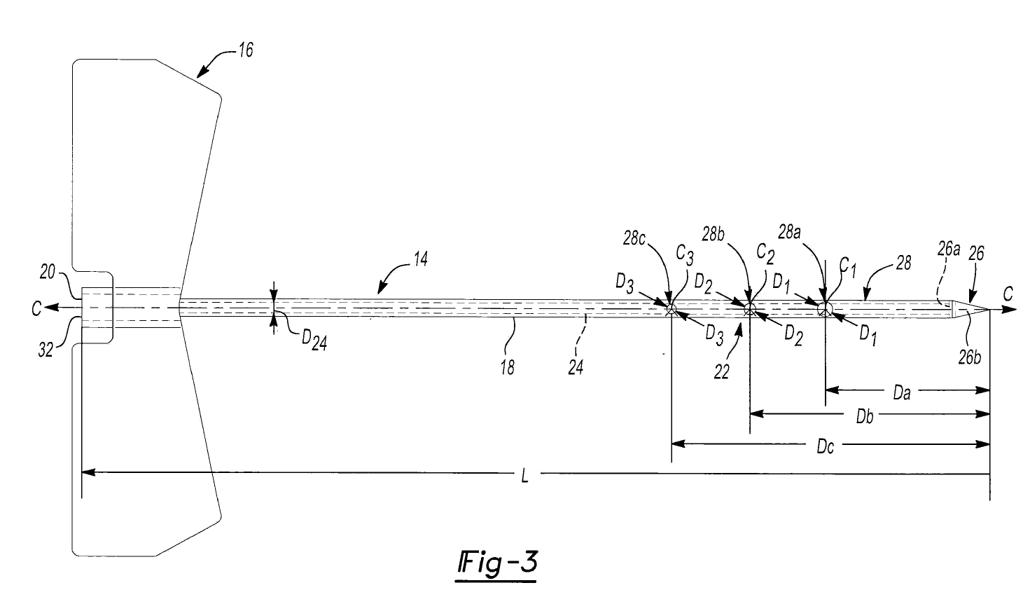

With additional reference to FIGS. 1 and 3, the cannulated body 14 can include an exterior surface 18, a proximal end 20, a distal end 22 and a bore 24 that can extend from the proximal end 20 to the distal end 22. The exterior surface 18 can be generally smooth to enable the cannulated body 14 to pass through the anatomy 12. The proximal end 20 can be coupled to the handle 16, and can be displaced from the distal end 22 at a length L. The length L can be sized such that the bone marrow aspiration needle 10 can be positioned at a desired depth into the anatomy 12, while still enabling a surgeon to grasp and manipulate the bone marrow aspiration needle 10 outside of the anatomy 12, as will be discussed (FIG. 2).

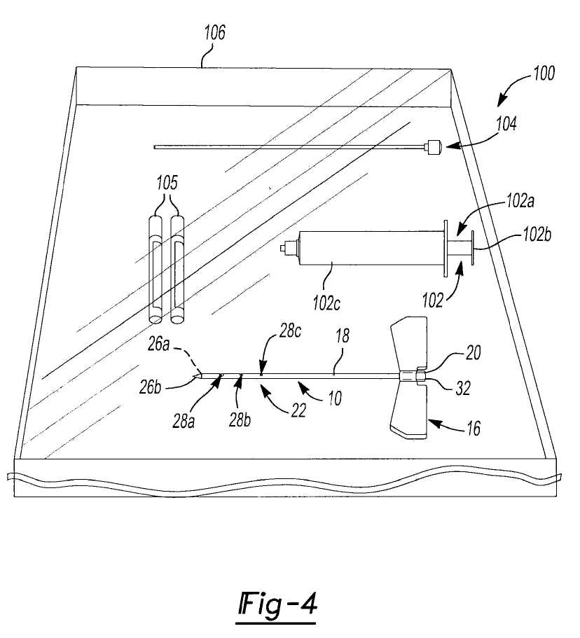

The distal end 22 can terminate at a tip 26. The tip 26 can have an open end 26a (as illustrated in phantom) for receipt of a stylet therethrough to assist in positioning the bone marrow aspiration needle 10 to the anatomy 12 (as will be discussed with reference to FIG. 4), or the tip 26 can include a piercing tip 26b. The open end 26a can further define a distal most aperture, if desired, such that the open end 26a can serve to harvest BMA 11 from the anatomy 12 (not specifically shown). As best shown in FIG. 2, the piercing tip 26b can penetrate a layer of skin 30 on the anatomy 12, along with the bone tissue 12a, to enable the surgeon to insert the bone marrow aspiration needle 10 into the anatomy 12.

With reference to FIGS. 1 and 3, the cannulated body 14 can define one or more apertures 28, and can generally define a first aperture 28a, a second aperture 28b and a third aperture 28c. It will be understood, however, that although the bone marrow aspiration needle 10 is illustrated herein as having three apertures 28, any number of apertures 28 could be employed to harvest the BMA 11 from the anatomy 12, and thus, the use of three apertures 28 is merely exemplary. Further, it should be noted that although the first aperture 28a, second aperture 28b and the third aperture 28c are illustrated herein as being circular in shape, the first aperture 28a, the second aperture 28b and the third aperture 28c can have any desired shape, such as oval, rectangular, square, starred, etc. In addition, it should be noted that although the first aperture 28a, second aperture 28b and third aperture 28c are illustrated herein as being defined on a single surface of the cannulated body 14, one or more of the first aperture 28a, second aperture 28b and third aperture 28c could extend through the cannulated body 14, and thus, could form bores through the cannulated body 14. Generally, the first aperture 28a, second aperture 28b and third aperture 28c can be formed on the cannulated body 14 by a post processing step, such as machining, punching, etc., but the first aperture 28a, second aperture 28b and third aperture 28c could be molded into the cannulated body 14, for example, if the cannulated body 14 is comprised of a composite or polymeric material.

The first aperture 28a can be defined at a first distance Da from the tip 26, and the second aperture 28b can be defined at a second distance Db from the tip 26. The third aperture 28c can be defined at a third distance Dc from the tip 26. Generally, the first distance Da can be less than the second distance Db, and in turn, the second distance Db can be less than the third distance Dc. Thus, relative to the cannulated body 14, the first aperture 28a can be defined as a distal aperture, and the second aperture or third aperture can be defined as a proximal aperture. In other words, the first aperture 28a can comprise a distal most aperture defined on the cannulated body 14, while the third aperture can comprise a proximal most aperture defined on the cannulated body 14.

Typically, the first aperture 28a, the second aperture 28b and the third aperture 28c can have corresponding center axes C₁, C₂, C₃ that can be perpendicular to and can intersect a central axis C of the cannulated body 14. It will be understood, however, that the first aperture 28a, the second aperture 28b and the third aperture 28c can be positioned offset from the central axis C of the cannulated body 14, such that one or more of the center axes C₁, C₂ and C₃ can be perpendicular to, but not necessarily intersect, the central axis C of the cannulated body 14 (as shown in phantom in FIG. 1).

In addition, the first aperture 28a can generally have a first surface area

$${A}_{1} ( {A}_{1} = {π ( \frac{{D}_{1}}{2} )}^{2} )$$

that is greater than a second surface area

$${A}_{2} ( {A}_{2} = {π ( \frac{{D}_{2}}{2} )}^{2} )$$

of the second aperture 28b, and the second surface area A₂ can be greater than a third surface area

$${A}_{3} ( {A}_{3} = {π ( \frac{{D}_{3}}{2} )}^{2} )$$

of the third aperture 28c. Insofar as the apertures 28a, 28b and 28c of the illustrated embodiment are circular, the first aperture 28a can have a diameter D₁ that is greater than a diameter D₂ of the second aperture 28b and the third aperture 28c can have a diameter D₃ that can be smaller than the diameter D₂ of the second aperture 28b. For example, for an 11-gage cannulated member 14, the diameter D₁ of the first aperture 28a can range from about 1.0 millimeter to about 2.5 millimeters, the diameter D₂ of the second aperture 28b can range from about 0.5 millimeters to about 2.0 millimeters, and the diameter D₃ of the third aperture 28c can range from about 0.25 millimeters to about 1.75 millimeters.

Generally, the surface areas A₁, A₂, A₃ of the first aperture 28a, second aperture 28b and third aperture 28c can decrease in size based on the distance Da, Db, Dc of the first aperture 28a, second aperture 28b and third aperture 28c from the tip 26 of the cannulated body 14, with the surface areas A₁, A₂, A₃ decreasing as the distances Da, Db, Dc increase.

Forming the cannulated body 14 with the surface areas A₁, A₂, A₃ decreasing as the distance from the tip 26 of the cannulated body 14 increases can serve to alter the fluid dynamics associated with the bone marrow aspiration needle 10 such that when a suction source is coupled to the bone marrow aspiration needle 10, equal volumes of BMA 11 are aspirated from each of the apertures 28, as will be discussed. In this regard, due to pressure gradients that exist within a cannulated member, such as a cannulated member of a bone aspiration needle, it can be difficult to aspirate an equal volume of BMA 11 from multiple locations in the anatomy 12 as the pressure gradients can enable the BMA 11 to be aspirated more rapidly through an aperture that is positioned more proximal to a handle of a bone aspiration needle. After the aspiration of 2-4 cubic centimeters (cc) of BMA 11 from the aperture near the bone aspiration needle, peripheral blood can become the primary material aspirated from this aperture and in effect can dilute the BMA 11 aspirated from the aperture.

The total volume of BMA 11 aspirated through each aperture 28 can be dependent upon the negative pressure and surface area A of each aperture 28. The pressure gradients present in a cannulated member, such as the cannulated body 14, can lead to a higher negative pressure and consequently a higher volumetric flow rate for an aperture 28 as it becomes more proximal to the handle 16. By providing the cannulated body 14 with apertures 28 that have surface areas A₁, A₂, A₃ that can be decreasing as the respective distance Da, Db, Dc from the tip 26 increase, the BMA 11 can be aspirated equally from each of the apertures 28 as the variation in surface areas A₁, A₂, A₃ can compensate for the different pressure gradients within the cannulated member 14. The variance in surface areas A can serve to counteract the different pressure gradients present in the cannulated member 14, and thus, can result in equal volumetric flow through all the apertures 28, regardless of the distance Da, Db, Dc of the respective aperture 28 from the tip 26.

In this regard, as the third aperture 28c has a smaller surface area A₃ than the second aperture 28b and first aperture 28a, the volumetric flow can be limited by the smaller surface area A₃, and therefore, a consistent volume of BMA 11 can be extracted, aspirated or harvested from the anatomy 12 adjacent to the third aperture 28c despite the high level of negative pressure in the cannulated member 14. This variance in surface areas A of the apertures 28 can enable the withdrawal of substantially equal amounts of the BMA 11 from the anatomy 12 at each aperture 28. Thus, the variation in the surface area A₁, A₂, A₃ of the first aperture 28a, second aperture 28b and third aperture 28c can enable a surgeon to aspirate a large volume of the BMA 11 from multiple harvest sites (i.e. the apertures 28) without repositioning the bone marrow aspiration needle 10 in the anatomy 12, as equal amounts of the BMA 11 can be extracted substantially simultaneously from each harvest site or aperture 28.

With continuing reference to FIGS. 1 and 3, the BMA 11, when harvested or aspirated through the apertures 28, can flow into the bore 24. The bore 24 can be defined from the tip 26 of the cannulated body 14 to the proximal end 20 of the cannulated body 14 to enable the BMA 11 to flow from the apertures 28 into a suction device positioned at the proximal end 20 of the bone marrow aspiration needle 10, as will be discussed further herein. The bore 24 can further have a diameter D₂₄ that is sized to enable the receipt of at least one surgical instrument, if desired, such as a stylet, as will be discussed. It should be noted that although the bore 24 is illustrated as extending from the tip 26 of the cannulated body 14 to the proximal end 20 of the cannulated body 14, the bore 24 can extend from the first aperture 28a to the proximal end 20 of the cannulated body 14, if desired, such as in the case of the tip 26 comprising a piercing tip 26a, for example.

The graspable portion or handle 16 can be coupled to the cannulated body 14. As the handle 16 can comprise any suitable handle that enables a surgeon or operator to manipulate the bone marrow aspiration needle 10, the handle 16 will not be discussed in great detail herein. Briefly, however, the handle 16 can be coupled to the proximal end 20 of the cannulated body 14, and be positioned about the cannulated body 14 such that at least one surgical instrument can be coupled to the bore 24 of the bone marrow aspiration needle 10 to enable the harvesting of the BMA 11. For example, the handle 16 can define an aperture 32 that can enable the handle 16 to be slid over the exterior surface 18 of the cannulated body 14, such that the handle 16 can then be coupled to the exterior surface 18 of the cannulated body 14 by press-fitting, mechanical fasteners, clamping, etc.

With reference now to FIG. 4, the bone marrow aspiration needle 10 can be part of a bone marrow aspiration kit 100. The kit 100 can include the bone marrow aspiration needle 10, a suction device 102 and an optional stylet 104. The kit 100 can also include one or more vials 105 for receipt of the harvested BMA 11, if desired. Various portions of the kit 100 can be inside at least one sterile container 106 prior to use. The sterile container 106 can comprise any suitable container that can maintain a sterile environment for the bone marrow aspiration needle 10, the suction device 102 and the optional stylet 104 and vials 105, and thus, the sterile container 106 will not be discussed in great detail herein. In addition, as the vials 105 can comprise any suitable sterile container, the vials 105 will not be discussed in great detail herein.

The suction device 102 can be coupled to the proximal end 20 of the cannulated body 14, and can apply a negative pressure or suction force to the bone marrow aspiration needle 10 to aspirate the BMA 11 from the anatomy 12 via the apertures 28. Generally, the suction device 102 can comprise a surgical syringe 102a as illustrated, however, any suitable suction device 102 can be used with the bone marrow aspiration needle 10. If the suction device 102 comprises a surgical syringe 102a, then the suction device 102 can be coupled to the proximal end 20 of the bone marrow aspiration needle 10 to harvest the BMA 11 from the anatomy 12 (as illustrated in FIG. 2). In the case of a surgical syringe 102a, a plunger 102b of the surgical syringe 102a can be pulled upward, away from a body 102c of the surgical syringe 102a to create a vacuum, negative pressure or suction force on the cannulated body 14. The vacuum, negative pressure or suction force can cause the BMA 11 to be withdrawn from the anatomy 12.

The stylet 104 can be optional, and if employed, can be used to pierce the anatomy 12. In this regard, if the bone marrow aspiration needle 10 includes a tip 26 with a piercing tip 26b, then the stylet 104 need not be included in the kit 100. If, however, the tip 26 comprises an open tip 26a, then the stylet 104 can be used in conjunction with the bone marrow aspiration needle 10 to position the bone marrow aspiration needle 10 within the anatomy 12. In order to position the bone marrow aspiration needle 10 in the anatomy 12, the stylet 104 can be inserted into the bore 24 such that the stylet 104 can extend beyond the open tip 26a of the cannulated body 14. The stylet 104 can then be used to pierce the anatomy 12, and a surgeon can apply a force necessary to drive the bone marrow aspiration needle 10 into the desired position in the anatomy 12 (not specifically shown). Once the bone marrow aspiration needle 10 is positioned in the desired location, the stylet 104 can be removed, and the suction device 102 can be coupled to the bone marrow aspiration needle 10 to harvest the BMA 11 from the anatomy 12.

Thus, in order to employ the bone marrow aspiration needle 10 to harvest the BMA 11 from an anatomy, such as an iliac crest 12a, the bone marrow aspiration needle 10 can be removed from the sterile package 106, if applicable. Then, either the piercing tip 26b of the cannulated body 14 can be used to drive the bone marrow aspiration needle 10 into the anatomy 12, or the stylet 104 can be inserted into the bore 24 of the cannulated body 14 and the stylet 104 can be used to drive the bone marrow aspiration needle 10 into the anatomy 12. Referring to FIG. 2, with the bone marrow aspiration needle 10 in the desired position in the anatomy 12, the suction device 102 can be coupled to the proximal end 20 of the cannulated body 14. Once the suction device 102 is coupled to the bone marrow aspiration needle 10, the suction device 102 can be actuated to apply a negative pressure or a suction force to the cannulated body 14. For example, the plunger 102b of the surgical syringe 102a can be pulled upward away from the body 102c of the surgical syringe 102a to create the negative pressure or suction force within the cannulated body 14.

With the suction device 102 activated such that the negative pressure or suction force is applied to the cannulated body 14, equal amounts of the BMA 11 can be withdrawn through each of the first aperture 28a, second aperture 28b and third aperture 28c. In this regard, as discussed, the variation between the surface areas A₁, A₂, A₃ can distribute the negative pressure among the apertures 28 such that the first aperture 28a, second aperture 28b and third aperture 28c can each draw equal amounts of the BMA 11 from the anatomy 12 adjacent to the first aperture 28a, second aperture 28b and third aperture 28c. The distribution of the surface areas A amongst the apertures 28 can serve to compensate for the pressure gradient that occurs within the bone marrow aspiration needle 10. In this regard, due to the fluid dynamics involved during the aspiration of the BMA 11 from the anatomy 12, a pressure gradient occurs that results in the amount of negative pressure applied to the apertures 28 decreasing from the aperture 28 that is proximal to the handle 16 (i.e. the third aperture 28c) to the aperture 28 that is distal from the handle 16 (i.e. the first aperture 28a). Thus, by adjusting the surface areas A of the apertures 28, the bone marrow aspiration needle 10 can compensate for the pressure gradient, and can thereby enable equal flow of BMA 11 through each of the apertures 28.

As the bone marrow aspiration needle 10 can withdraw equal amounts of the BMA 11 from each harvest site in the anatomy 12 substantially simultaneously, the bone marrow aspiration needle 10 can harvest about 6 cc to about 12 cc of BMA on each insertion into the anatomy 12 (about 2 cc to about 4 cc of BMA 11 per aperture 28). As most bone graft procedures require more than about 4 cc of the BMA 11, the use of the bone marrow aspiration needle 10 can reduce the need for the surgeon to puncture the anatomy 12 multiple times to obtain the required amount of BMA 11 for the bone graft.

While specific examples have been described in the specification and illustrated in the drawings, it will be understood by those of ordinary skill in the art that various changes can be made and equivalents can be substituted for elements thereof without departing from the scope of the present disclosure as defined in the claims. Furthermore, the mixing and matching of features, elements and/or functions between various examples is expressly contemplated herein so that one of ordinary skill in the art would appreciate from this disclosure that features, elements and/or functions of one example can be incorporated into another example as appropriate, unless described otherwise, above. Moreover, many modifications can be made to adapt a particular situation or material to the teachings of the present disclosure without departing from the essential scope thereof. Therefore, it is intended that the present disclosure not be limited to the particular examples illustrated by the drawings and described in the specification as the best mode presently contemplated for carrying out this invention, but that the scope of the present disclosure will include any embodiments falling within the foregoing description and the appended claims.

1. A bone aspiration needle comprising:
a cannulated body defining at least a first aperture for aspirating bone marrow from a first region of an anatomy, a second aperture for aspirating bone marrow from a second region of the anatomy and a third aperture for aspirating bone marrow from a third region of the anatomy, the first aperture being a distal most aperture and the third aperture being a proximal most aperture; and
a suction device in communication with the proximal end of the cannulated body that applies a negative pressure to the cannulated body to aspirate at least four cubic centimeters of bone marrow from a single insertion site in the anatomy from the first region, second region and third region of the anatomy such that the aspirated bone marrow flows into the suction device;
wherein the first aperture has a first area, the second aperture has a second area, and the third aperture has a third area, the second area being less than the first area and greater than the third area.

2. The bone aspiration needle of claim 1, wherein the cannulated body has a proximal end and a distal end that includes a tip, and the first aperture, second aperture and third aperture are defined proximate in the distal end of the cannulated body, with the first aperture being defined proximate the tip.

3. The bone aspiration needle of claim 2, further comprising a graspable portion coupled to the proximal end of the cannulated body.

4. (canceled)

5. The bone aspiration needle of claim 1, wherein the suction device is operable to aspirate substantially equal amounts of bone marrow substantially simultaneously from each of the first aperture, second aperture and third aperture.

6. (canceled)

7. The bone aspiration needle of claim 2, wherein the tip comprises a piercing tip to enable the bone aspiration needle to be driven into the anatomy.

8. The bone aspiration needle of claim 2, further comprising a stylet that includes a piercing tip that is operable to extend beyond the tip of the cannulated body to enable the bone aspiration needle to be driven into the anatomy.

9. The bone aspiration needle of claim 1, wherein the cannulated body has a central axis, the first aperture, second aperture and third aperture each have a center axis, and the center axis of at least one of the first aperture, second aperture and third aperture is perpendicular to and offset from the central axis of the cannulated body.

10. The bone aspiration needle of claim 1, wherein the cannulated body has a central axis, the first aperture, second aperture and third aperture each have a center axis, and the center axis of at least one of the first aperture, second aperture and third aperture is perpendicular to and intersects the central axis of the cannulated body.

11. The bone marrow aspiration needle of claim 2, wherein the first aperture is defined at a first distance from the tip, the second aperture is defined at a second distance from the tip and the third aperture is defined at a third distance from the tip, with the second distance being greater than the first distance and less than the third distance.

12. A bone aspiration needle comprising:
a cannulated body defining at least a distal aperture for aspirating bone marrow from a first region of an anatomy and a proximal aperture for aspirating bone marrow from a second region of the anatomy; and
a suction device in communication with the cannulated body that applies a negative pressure to the cannulated body to aspirate at least four cubic centimeters of bone marrow from the first region and second region of the anatomy at a single insertion site for the bone aspiration needle in the anatomy;
wherein the distal aperture has a first area and the proximal aperture has a second area, with the second area being less than the first area such that the aspirated bone marrow from the first region and the second region flows through a central bore of the cannulated body, with both the distal aperture and the proximal aperture in direct communication with the central bore.

13. The bone aspiration needle of claim 12, wherein the cannulated body has a proximal end and a distal end that includes a tip, and the distal aperture and the proximal aperture are defined in the distal end of the cannulated body, with the distal aperture being defined adjacent to the tip.

14. The bone aspiration needle of claim 13, further comprising a graspable portion coupled to the proximal end of the cannulated body.

15. The bone aspiration needle of claim 13, wherein the cannulated body further defines a third aperture for aspirating bone marrow from a third region of the anatomy, third aperture being proximal to the second aperture and having a third area that is less than the second area, the suction device being operable to aspirate substantially equal amounts of bone marrow from each of the first aperture, second aperture and third aperture.

16. The bone aspiration needle of claim 13, wherein the tip comprises a piercing tip to enable the bone aspiration needle to be driven into the anatomy.

17. The bone aspiration needle of claim 13, further comprising a stylet that includes a piercing tip that is operable to extend beyond the tip of the cannulated body to enable the bone aspiration needle to be driven into the anatomy.

18. The bone aspiration needle of claim 15, wherein the cannulated body has a central axis, the first aperture, second aperture and third aperture each have a center axis, and the center axis of at least one of the first aperture, second aperture and third aperture is perpendicular to and intersects the central axis of the cannulated body.

19. A bone aspiration needle comprising:
a cannulated body having a proximal end and a distal end that includes a tip, the cannulated body defining a central bore, at least a first aperture for aspirating bone marrow from a first region of an anatomy, a second aperture for aspirating bone marrow from a second region of the anatomy and a third aperture for aspirating bone marrow from a third region of the anatomy, the first aperture being proximal to the tip, the third aperture being distal from the tip, the second aperture being disposed between the first aperture and the third aperture, and each of the first aperture, second aperture and third aperture in direct communication with the central bore; and
a suction device in communication with the proximal end of the cannulated body applies a negative pressure to the cannulated body to aspirate at least two cubic centimeters of bone marrow from each of the first region, second region and third region of the anatomy at a single insertion site for the bone aspiration needle in the anatomy;
wherein the first aperture has a first area, the second aperture has a second area and the third aperture has a third area, with the second area being less than the first area and greater than the third area such that the suction device aspirates substantially equal amounts of bone marrow from the first aperture, the second aperture and the third aperture, and the aspirated bone marrow flows from each of the first aperture, second aperture and third aperture into the central bore.

20. The bone aspiration needle of claim 19, further comprising a graspable portion coupled to the proximal end of the cannulated body.

21. (canceled)

22. A method of using the bone aspiration needle of claim 1, comprising:
inserting the cannulated body into the anatomy;
coupling the suction device to the proximal end of the cannulated body;
actuating the suction device to apply the negative pressure to the cannulated body;
aspirating the at least four cubic centimeters of bone marrow from the single insertion site; and
withdrawing the bone marrow from cannulated body into the suction device.

23. The method of claim 22, wherein aspirating includes:
aspirating at least two cubic centimeters of bone marrow from the first region of the anatomy via the first aperture;
aspirating at least two cubic centimeters of bone marrow from the second region of the anatomy via the second aperture; and
aspirating at least two cubic centimeters of bone marrow from the third region of the anatomy via the third aperture.
